(19) United States
(12) Patent Application Publication
(10) Pub. No.: US 2022/0296138 A1
(43) Pub. Date: Sep. 22, 2022

(54) SYSTEMS AND METHODS FOR EVALUATING THE EFFICACY OF MEDICAL TREATMENT(S) FOR ADHD

(71) Applicant: iFocus Health Inc., San Jose, CA (US)

(72) Inventors: Adi Diner, San Jose, CA (US); Einat Sitbon, Ness Ziona (IL); Yanxia Zhang, Foster City, CA (US); Yulie Klerman, San Francisco, CA (US); Reem Yunis, Menlo Park, CA (US)

(21) Appl. No.: 17/686,589

(22) Filed: Mar. 4, 2022

Related U.S. Application Data
(60) Provisional application No. 63/162,801, filed on Mar. 18, 2021.
(60) Provisional application No. 63/307,798, filed on Feb. 8, 2022.

(51) Int. Cl. A61B 5/16; G16H 20/70; G16H 50/20; G16H 40/63; A61B 5/0205; A61B 5/00

(52) U.S. Cl. CPC A61B 5/168; G16H 20/70; G16H 50/20; G16H 40/63; A61B 5/163; A61B 5/0205; A61B 5/4848; A61B 5/7267; A61B 5/024

(57) ABSTRACT

A system for evaluating a treatment for a mental or physiological condition. The system includes a sensor configured to measure eye movements of a user, a processor, and a memory. The memory includes instructions, which, when executed by the processor, cause the system to: identify a task; prior to the treatment and while the user performs the task, measure the eye movements of the user with the sensor; while the user is under an effect of the treatment and while the user performs the task, measure eye movements of the user with the sensor; determine a difference between the eye movements of the user prior to and after the treatment based on a trained machine learning model; determine a measure of an efficacy of the treatment based on the determined difference in the measured eye movements of the user; and display a recommended a course of treatment based on the determined measure of efficacy.

### CROSS-REFERENCE TO RELATED APPLICATIONS

The present application claims the benefit of U.S. Provisional Patent Application No. 63/162,801, filed on Mar. 18, 2021, and U.S. Provisional Patent Application No. 63/307,798, filed on Feb. 8, 2022, the entire contents of each of which are hereby incorporated herein by reference.

### TECHNICAL FIELD

This disclosure relates to systems and methods for evaluating the efficacy of treatment for mental and/or neurological disorders and conditions.

### BACKGROUND

Attention deficit hyperactivity disorder (ADHD) is a neurodevelopmental disorder characterized by inattention, or excessive activity and impulsivity, which are otherwise not appropriate for a person's age. ADHD is a chronic condition that affects millions of children and often continues into adulthood. ADHD may lead to a combination of persistent problems, such as difficulty sustaining attention, hyperactivity, and impulsive behavior.

Children with ADHD may also struggle with low self-esteem, troubled relationships, and poor performance in school. Symptoms sometimes lessen with age. However, some people may still exhibit ADHD symptoms as they become adults. In some cases, people with ADHD may be able to learn strategies to compensate for or otherwise lessen the impact of their symptoms. In addition, there are treatments that can assist in coping with ADHD, such as medications and behavioral interventions.

However, a concern regarding present treatments and approaches for people coping with ADHD is that conventional approaches typically are focused on testing someone to determine if they have ADHD (e.g., as a form of diagnosis) by comparing their performance on a task to that of a general population. This approach, while sometimes useful, may fail to properly diagnose someone who has learned to perform the task competently. Further, due to the complex nature of ADHD and its various symptoms and how those may be manifested in a person, this approach may not be useful for monitoring the impact of treatments as each person's experience with ADHD and response to treatment may differ.

What is desired are systems, apparatuses, and methods for more accurately and effectively determining the impact of a treatment for ADHD on a specific person. Aspects of the disclosure described herein address this and other objectives both individually and collectively.

### SUMMARY

According to one aspect of this disclosure, a system for evaluating a treatment for a mental or physiological condition. The system includes a sensor configured to measure eye movements of a user, a processor, and a memory. The memory includes instructions, which, when executed by the processor, cause the system to: identify a task; prior to the treatment of the user and while the user performs the task, measure the eye movements of the user with the sensor; while the user is under an effect of the treatment of the user and while the user performs the task, measure eye movements of the user with the sensor; determine a difference between the eye movements of the user prior to and after the treatment of the user based on a trained machine learning model; determine a measure of efficacy of the treatment of the user based on the determined difference in the measured eye movements of the user; and display a recommended a course of treatment for the user based on the determined measure of efficacy.

In an aspect of the present disclosure, the system may further include a second sensor configured to measure a biomarker. The instructions, when executed by the processor, may further cause the system to measure, by the second sensor, the biomarker of the user while the user performs the task and prior to the treatment of the user; and measure, by the second sensor, the biomarker of the user while performing the task after the treatment.

In another aspect of the present disclosure, the instructions, when executed by the processor, may further cause the system to determine a difference between the measured biomarker of the user prior to and after the treatment of the user.

In yet another aspect of the present disclosure, determining the measure of the efficacy of the treatment of the user may be further based on the difference in the measured biomarker of the user.

In a further aspect of the present disclosure, the biomarker may include a heart rate, a head movement, and/or fidgeting of the user.

In yet a further aspect of the present disclosure, the task may include a section of text displayed on a screen for the user to read.

In an aspect of the present disclosure, the sensor may include an eye-tracking device.

In another aspect of the present disclosure, the instructions, when executed by the processor, may further cause the system to determine the measure of the efficacy of the treatment of the user based on the determined difference in the measured eye movements of the user by inputting data relating to the eye movements of the user into the trained machine learning model, the trained machine learning model configured to generate an output representing a measure of an improvement and/or deterioration to an ability of the user to perform the task due to the treatment of the user. The improvement and/or deterioration is relative to the measured eye movements of the user prior to the treatment of the user.

In yet another aspect of the present disclosure, the data regarding the eye movements may include a total amplitude of saccade and number of saccades during the performance of the task.

According to another aspect, a computer-implemented method for evaluating a treatment for a condition of a user is presented. The computer-implemented method includes accessing a first data set indicating a pretreatment condition; accessing a second data set indicating a post-treatment condition; predicting a measure of efficacy of the treatment of the user by a trained machine learning model based on the first data set and the second data set; and displaying a recommended course of treatment for the user based on the predicted measure of efficacy.

In yet another aspect of the present disclosure, the first data set may include measured eye movements of the user while the user performs a task and prior to the treatment of the user. The second data set may include measured eye movements of the user while the user performs the task after the treatment of the user.

In a further aspect of the present disclosure, the first data set may further include a measured biomarker of the user while the user performs the task and prior to the treatment. The second data set may include the measured biomarker of the user while the user performs the task after the treatment.

In an aspect of the present disclosure, the computer-implemented method may further include determining a difference between the measured biomarker of the user prior to and after the treatment of the user.

In another aspect of the present disclosure, predicting the measure of the efficacy of the treatment of the user may be further based on the difference in the measured biomarker of the user.

In yet another aspect of the present disclosure, the biomarker may include a heart rate, a head movement, and/or fidgeting of the user.

In a further aspect of the present disclosure, the task may include a section of text displayed on a screen for the user to read.

In yet a further aspect of the present disclosure, the eye movements of the user may be measured using an eye-tracking device.

In an aspect of the present disclosure, the computer-implemented method may further include predicting the measure of efficacy of the treatment of the user by the machine learning model based on the first data set and the second data set by inputting data relating to eye movements into the trained machine learning model. The trained model may be configured to generate an output representing a measure of change in an ability of the user to perform the task due to the treatment of the user. The change compared to the measured eye movements of the user prior to the treatment of the user.

In another aspect of the present disclosure, the computer-implemented method may further include comparing the prediction to a third data set indicative of recommended courses of treatment and determining the recommended course of treatment based on the comparison to the third data set.

According to another aspect, a computer-implemented method for evaluating a treatment for a condition, the computer-implemented method comprising capturing a first data set indicating a performance of a task by a user prior to treatment of the user, capturing a second data set indicating a performance of the task by the user after treatment of the user, predicting a measure of efficacy of the treatment of the user by a trained machine learning model based on the first data set and the second data set, and displaying a recommended course of treatment for the user based on the predicted measure of efficacy.

Other aspects, features, and advantages will be apparent from the description, the drawings, and the claims that follow.

### BRIEF DESCRIPTION OF DRAWINGS

The accompanying drawings, which are incorporated in and constitute a part of this specification, illustrate aspects of the disclosure and, together with a general description of the disclosure given above and the detailed description given below, explain the principles of this disclosure, wherein:

FIG. 1 is a diagram of a system for methods for evaluating the efficacy of medical treatment(s) for a mental and/or neurological condition;

FIG. 2 is a flow diagram illustrating a computer-implemented method for use with the system of FIG. 1;

FIG. 3 is a flow diagram illustrating a computer-implemented method for use with the system and methods of FIG. 1;

FIG. 4A is a diagram illustrating an example of a displayed paragraph shown as a pretreatment task for a user to perform for the systems and methods of FIG. 1, the diagram having a circled area within the display to illustrate a fixation point of the user's pretreatment focus, the diagram also illustrating an elongated line extending across displayed words of the displayed paragraph and representing a pretreatment saccade of the user;

FIG. 4B is a diagram illustrating an example of a displayed paragraph shown as a post-treatment task for the user to perform for the systems and methods of FIG. 1, the displayed paragraph illustrating the user's fixation area and saccade post-treatment;

FIG. 5 is a diagram illustrating an exemplary report generated by the system of FIG. 1, in accordance with the disclosure;

FIG. 6 is a graphical representation of reading slope with and without user treatment;

FIG. 7 is a graphical representation of user page re-reading versus user gaze with and without user treatment;

FIG. 8 is a graphical representation of user gaze on each page position with and without user treatment;

FIG. 9 is a graphical representation of time to first saccade with and without user treatment; and

FIG. 10 is a graphical representation of a maximum peak velocity of saccades with and without user treatment.

### DETAILED DESCRIPTION

Aspects of the disclosed systems are described in detail with reference to the drawings, in which like reference numerals designate identical or corresponding elements in each of the several views. As used herein, the term “clinician” refers to a doctor, a nurse, or any other care provider and may include support personnel. In the following description, well-known functions or constructions are not described in detail to avoid obscuring the present disclosure in unnecessary detail.

This disclosure relates to systems and methods for evaluating the efficacy of treatment for mental and/or neurological disorders and conditions.

Among other things, the present disclosure may be embodied in whole or in part as a system, as one or more methods, or as one or more devices. Aspects of the disclosure may take the form of a hardware-implemented aspect, a software-implemented aspect, or an aspect combining software and hardware aspects. For example, in some aspects, one or more of the operations, functions, processes, or methods described herein may be implemented by one or more suitable processing elements (such as a processor, microprocessor, CPU, GPU, TPU, controller, etc.) that is part of a client device, server, network element, remote platform (such as a SaaS platform), an “in the cloud” service, or other form of computing or data processing system, device, or platform.

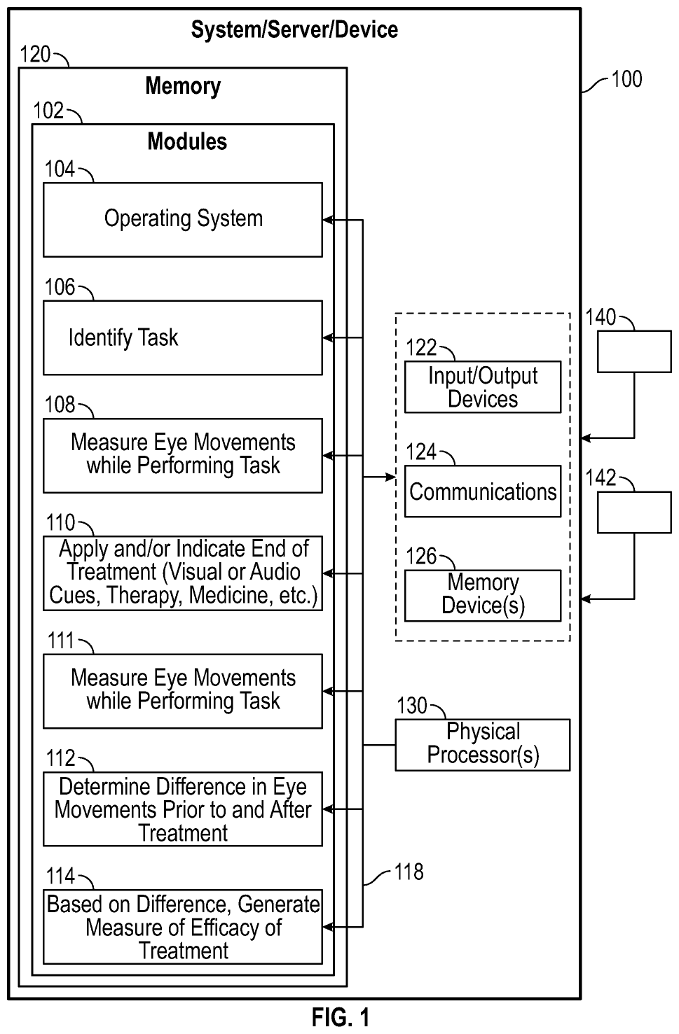

Referring to FIG. 1, a system 100 for evaluating the efficacy of treatment for mental and/or neurological disorders and conditions is shown. The system 100 generally includes a sensor 140 configured for measuring eye movements of a user (e.g., a patient) while performing a task, a processor 130, and a memory 120 that contains instructions for execution by the processor 130.

In aspects, the system 100 may represent a server or other form of computing or data processing system, platform, or device. Modules 102 each contain a set of executable instructions, where when the set of instructions is executed by a suitable electronic processor or processors (such as that indicated in the figure by “Physical Processor(s) 130”), system (or server, platform, or device) 100 operates to perform a specific process, operation, function, or method. Modules 102 are stored in memory 120, which typically includes an Operating System module 104 that contains instructions used (among other functions) to access and control the execution of the instructions contained in other modules. The modules 102 stored in memory 120 are accessed for purposes of transferring data and executing instructions by use of a “bus” or communications line 118, which also serves to permit processor(s) 130 to communicate with the modules for purposes of accessing and executing a set of instructions. Bus or communications line 118 also permits processor(s) 130 to interact with other elements of system 100, such as input or output devices 122, communications elements 124 for exchanging data and information with devices external to system 100, and additional memory devices 126.

For example, Identify Task Module 106 may contain computer-executable instructions which, when executed by a processor, cause the processor or a device in which it is implemented to identify a task. The identified task may be everyday tasks that the user is already performing, for example, reading a section of text. The Identify Task Module 106 may identify equivalent sections of text as a baseline pre-treatment task and/or as a post-treatment task. The identified task may be based on one or more of the user's demographics, status with regards to a neurological and/or mental disorder (such as attention deficit hyperactivity disorder (ADHD) and/or Attention Deficit Disorder (ADD)), the user's prior testing experiences, an expert's recommendation, etc. In some aspects, module 106 may contain instructions that, when executed, generate a user interface to allow selection of a desired test or evaluation protocol. Although ADHD is used as an illustrative example, the systems and methods of the disclosure are applicable to other neurological and/or mental disorders (such as bipolar disorder, depression, etc.).

Measure Eye Movements While Performing Task Module 108 may contain computer-executable instructions which, when executed by a processor, cause the processor or a device in which it is implemented to obtain data from an eye-tracking device (camera, phone, image capturing device, etc.) as the user performs the selected task. This may include sensing or detecting eye movement, tracking eye movement, and processing the data representing the eye position or movement as the task is performed. The processing of the data may produce a graph, line track, map, or other indication of eye fixation, dwell time, and eye movement.

Apply and/or Indicate End of Treatment (Visual or Audio Cues, Therapy, Medicine, etc.) Module 110 may contain computer-executable instructions which, when executed by a processor, cause the processor or a device in which it is implemented to implement or assist in implementing a specified “treatment” for the user, or receive an indication that a treatment has been completed. As examples, a treatment in this context might comprise administration of medication, generation of a visual cue (such as lighting), generation of an audio cue (nighttime sounds, peaceful sounds, etc.), or setting of a timer while the user undergoes an exercise routine. In some aspects, the treatment may be initiated or performed wholly by a person, either a caregiver or the person being tested. In some aspects, module 110 may contain instructions which when executed generate a user interface to allow entry of an indication that a treatment has been completed.

Note that the described methodology is “agnostic” with regards to a specific treatment, in that it may be used with any form of treatment that a person utilizes or that is provided for the person (including, but not limited to or requiring, medication, physical therapy, visual or audio cues, meditation, exercise, placement in a specific environment or location, undergoing a specific experience, etc.). The methodology does not typically select the treatment, although data collected through use of the system, apparatuses, and methods described may be used in generating a recommended treatment or modification to a treatment (such as by use of a trained machine learning model that classifies eye movement data or eye movement difference data and an applied treatment to generate an output representing how to modify or replace the treatment to obtain eye movement data more similar to someone with a lesser degree of ADHD, for example).

Measure Eye Movements While Performing Task Module 111 will, in some aspects, contain a similar or the same set of instructions as Module 108; that is, the instructions will operate to cause a processor or device to obtain data from an eye-tracking device (camera, phone, image capturing device, etc.) as the user performs the selected task.

Determine Difference in Eye Movements Prior to and After Treatment Module 112 may contain computer-executable instructions, which when executed by a processor (such as processor 130), cause the processor or a device in which it is implemented to determine a difference between the eye movement data obtained prior to (Module 108) and after (Module 111) the treatment. This “difference” may be represented as a point-to-point difference in eye position as a function of time, a comparison of a number or metric derived from the eye-tracking data for each situation (average dwell time, average number of certain events, whether certain behaviors are present or absent, etc.). In some aspects, the difference may be obtained by subtracting a graph or function (such as a polynomial or other form of curve) representing the user's eye movements prior to the treatment from a similar graph or function based on data obtained after the treatment. Based on Difference, Generate Measure of Efficacy of Treatment Module 114 may contain computer-executable instructions which, when executed by a processor, cause the processor or a device in which it is implemented to, based on the difference data or metric obtained from execution of the instructions contained in Module 112, generate a measure of the effectiveness of the treatment. In some aspects, this may be a number, a range of numbers, a measure of a standard deviation from a norm for a treatment, a measure of how effective the treatment was compared to other treatments for the user, etc. In some aspects, the eye movement or eye-tracking difference data may be provided as an input to a trained machine learning model to generate the efficacy measure. In some aspects, the eye movement or eye-tracking difference data may be provided as an input to an algorithm, rule-set, or form of statistical analysis to generate the efficacy measure.

Note that a possible source of revenue is to use the product as described herein to support pharmaceutical clinical trials. In this use case, it is used to measure treatment efficacy, not in order to directly drive treatment decisions for the specific user, but rather to determine or measure the effectiveness of a drug. Note that aspects may include questions regarding side effects, sleep patterns, and other relevant subjects. This information may be used together with the eye-tracking information collected from a user in order to provide a clinician with another source of data. A useful output may be represented as “the presumed efficacy of the treatment is X, and at this dose or level of treatment, the user is experiencing side effects of a type A with a severity Y.”

It should be understood that the present technology, as described above, can be implemented in the form of control logic using computer software in a modular or integrated manner. Based on the disclosure and teachings provided herein, a person of ordinary skill in the art will know and appreciate other ways and/or methods to implement the present technology using hardware and a combination of hardware and software.

In some aspects, certain of the methods, models, or functions described herein may be embodied in the form of a trained neural network, where the network is implemented by the execution of a set of computer-executable instructions or representation of a data structure. The instructions may be stored in (or on) a non-transitory computer-readable medium and executed by a processor or processing element. The set of instructions may be conveyed to a user through a transfer of instructions or an application that executes a set of instructions (such as over a network, e.g., the Internet). The set of instructions or an application may be utilized by an end-user through access to a SaaS platform or a service provided through such a platform. A trained neural network, trained machine learning model, or other form of decision or classification process may be used to implement one or more of the methods, functions, processes, or operations described herein. Note that a neural network or deep learning model may be characterized in the form of a data structure in which are stored data representing a set of layers containing nodes, and connections between nodes in different layers are created (or formed) that operate on an input to provide a decision or value as an output.

In general terms, a neural network may be viewed as a system of interconnected artificial “neurons” that exchange messages between each other. The connections have numeric weights that are “tuned” during a training process, so that a properly trained network will respond correctly when presented with an image or pattern to recognize (for example). In this characterization, the network consists of multiple layers of feature-detecting “neurons”; each layer has neurons that respond to different combinations of inputs from the previous layers. Training of a network is performed using a “labeled” dataset of inputs in a wide assortment of representative input patterns that are associated with their intended output response. Training uses general-purpose methods to iteratively determine the weights for intermediate and final feature neurons. In terms of a computational model, each neuron calculates the dot product of inputs and weights, adds the bias, and applies a non-linear trigger or activation function (for example, using a sigmoid response function).

Machine learning (ML) may be used to enable the analysis of data and assist in making decisions. In order to benefit from using machine learning, a machine learning algorithm is applied to a set of training data and labels to generate a “model” which represents what the application of the algorithm has “learned” from the training data. Each element (or example, in the form of one or more parameters, variables, characteristics or “features”) of the set of training data is associated with a label or annotation that defines how the element should be classified by the trained model. A machine learning model predicts a defined outcome based on a set of features of an observation. The machine learning model is built by training on a dataset which includes features and known outcomes. There are various types of machine learning algorithms, including linear models, support vector machines (SVM), random forest, and/or XGBoost. A machine learning model may include a set of layers of connected neurons that operate to decide (such as a classification) regarding a sample of input data. When trained (e.g., the weights connecting neurons have converged and become stable or within an acceptable amount of variation), the model will operate on a new element of input data to generate the correct label or classification as an output. Any other suitable machine learning model may be used.

For example, an algorithm and/or machine learning model may be used to process captured eye tracker data and analyze the captured data. A post treatment measurement will be compared to the user's baseline measurement retrieved in several different sessions in order to eliminate irrelevant variables (such as environment or fatigue). The comparison between a user's performance on the equivalent task pre- and post-treatment may be converted into a metric or other form of evaluating the efficacy or impact of the treatment. Repeating the test will improve the outcome reliability.

Any of the software components, processes, or functions described in this application may be implemented as software code to be executed by a processor using any suitable computer language such as Python, R, Java, JavaScript, C++, or Perl using conventional or object-oriented techniques. The software code may be stored as a series of instructions, or commands in (or on) a non-transitory computer-readable medium, such as a random-access memory (RAM), a read only memory (ROM), a magnetic medium such as a hard-drive or a floppy disk, or an optical medium such as a CD-ROM. In this context, a non-transitory computer-readable medium is almost any medium suitable for the storage of data or an instruction set aside from a transitory waveform. Any such computer readable medium may reside on or within a single computational apparatus and may be present on or within different computational apparatuses within a system or network.

According to one example implementation, the term processing element or processor, as used herein, may be a central processing unit (CPU), or conceptualized as a CPU (such as a virtual machine). In this example implementation, the CPU, or a device in which the CPU is incorporated may be coupled, connected, and/or in communication with one or more peripheral devices, such as display. In another example implementation, the processing element or processor may be incorporated into a mobile computing device, such as a smartphone or tablet computer.

The non-transitory computer-readable storage medium referred to herein may include a number of physical drive units, such as a redundant array of independent disks (RAID), a floppy disk drive, a flash memory, a USB flash drive, an external hard disk drive, thumb drive, pen drive, key drive, a High-Density Digital Versatile Disc (HD-DV D) optical disc drive, an internal hard disk drive, a Blu-Ray optical disc drive, or a Holographic Digital Data Storage (HDDS) optical disc drive, synchronous dynamic random access memory (SDRAM), or similar devices or other forms of memories based on similar technologies. Such computer-readable storage media allow the processing element or processor to access computer-executable process steps, application programs and the like, stored on removable and non-removable memory media, to off-load data from a device or to upload data to a device. As mentioned, with regards to the aspects described herein, a non-transitory computer-readable medium may include almost any structure, technology, or method apart from a transitory waveform or similar medium.

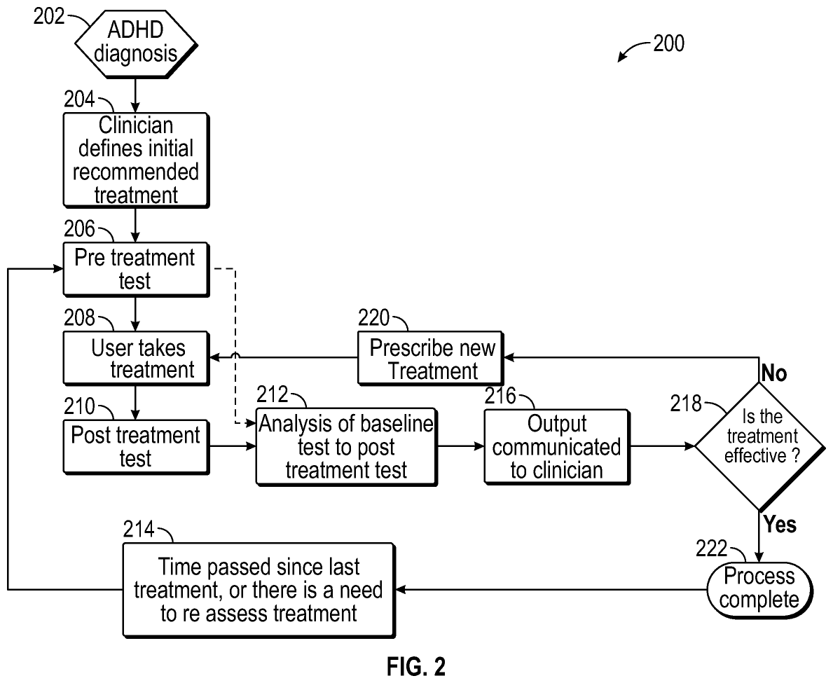

FIG. 2 shows a flow chart illustrating the various operations of an exemplary method for method for evaluating the efficacy of treatment for mental and/or neurological disorders and conditions. Persons skilled in the art will appreciate that one or more operations of the method 200 may be performed in a different order, repeated, and/or omitted without departing from the scope of the disclosure. In various aspects, the illustrated method 200 can operate on processor 130 (FIG. 1), in a remote device, or in another server or system. Other variations are contemplated to be within the scope of the disclosure. The operations of method 200 will be described with respect to a controller, e.g., processor 130 (FIG. 1) of system 100 (FIG. 1), but it will be understood that the illustrated operations are applicable to other systems and components thereof as well.

The processing element or elements may be programmed with a set of executable instructions (e.g., software instructions), where the instructions may be stored on (or in) one or more suitable non-transitory data storage elements. In some aspects, the set of instructions may be conveyed to a user through a transfer of instructions or an application that executes a set of instructions (such as over a network, e.g., the Internet). In some aspects, a set of instructions or an application may be utilized by an end-user through access to a SaaS platform or a service provided through such a platform.

In some aspects, one or more of the operations, functions, processes, or methods described herein may be implemented by a specialized form of hardware, such as a programmable gate array, application specific integrated circuit (ASIC), or the like. Note that an aspect of the methods of the present disclosure may be implemented in the form of an application, a sub-routine that is part of a larger application, a “plug-in,” an extension to the functionality of a data processing system or platform, or other suitable form. In aspects, the operations, functions, processes, or methods described herein may be implemented on a web or mobile application. The following detailed description is, therefore, not to be taken in a limiting sense.

A clinician may define an initial recommended treatment (204) for a user that has a diagnosis for a mental and/or neurological disorder, such as ADHD (202). As used herein, treatment is not limited to clinical treatment, and may include any action taken to improve performance. For example, taking medication, avoiding sugar, exercise, etc.

Initially, at step 206, the processor 130 captures a first data set indicating a performance of a task by a user (e.g., a patient) prior to treatment (e.g., a baseline test). For example, the task may include reading a section of text displayed on a screen. People with ADHD, for example, often read faster with treatment by comparison to when reading without treatment. A proposed reason for the lower reading ability and speed of users with such condition(s) is the need to re-read the equivalent region in a given text. The first dataset may be captured using a sensor 140 (e.g., an eye-tracking device). In aspects, the sensor 130 may capture data as a background process while the user is performing daily tasks, which may include reading.

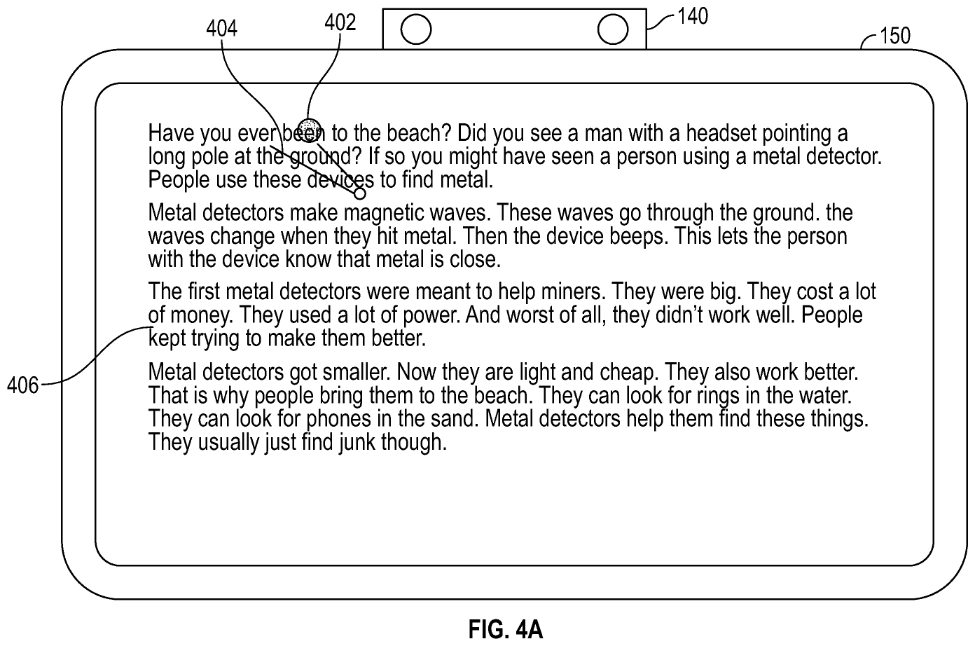

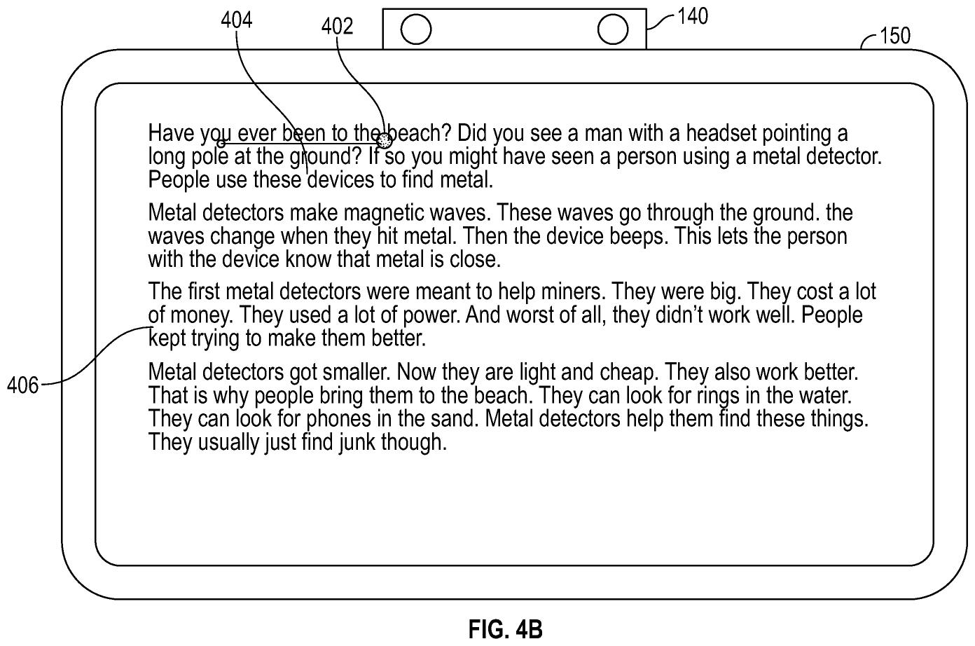

The first data set may include eye-tracking data, such as the total amplitude of saccade and number of saccades during performance of the task, pretreatment. A saccade is the rapid movement of the eye between two or more fixation points. The eye-tracking may be captured using sensor 140, which is configured to track eye movements of a user (e.g., an eye-tracking device and/or other imaging device). For example, the user (e.g., a patient) may read a passage (FIG. 4A) prior to treatment, and the processor 130 will measure the user's eye movements while reading the passage, using an eye-tracking device (e.g., sensor 140). Other examples of sensors may include smartwatches (e.g., devices with accelerometers, magnetometers, heart rate monitors, etc.) and/or web cams.

Eye movement or eye-tracking data may be collected during the task (e.g., test) using a suitable eye tracker hardware. The eye tracker may be installed on a computer or other device to track the eye movements of the user of the computer (or another device) who is viewing a screen or display thereof. In other aspects, the task may use a webcam, cell phone camera, or other form of camera or image capture device to track and record eye movements.

The task may be one in which a user (e.g., the patient) of a computer or other device is asked to view something being displayed on the computer/device screen (e.g., display 150). The display may be of a section of text to be read, a set of images to view (in some cases, perhaps with an associated request to select one in response to a question), a request to enter certain words or numbers using a keyboard, a request to respond to an image by a specific eye movement, etc. The task may be stand-alone or have follow-up tasks, such as answering questions related to the text or selecting specific images. The task may include one or more tasks. The tasks are presented on the computer/tablet/cell phone screen, and during each task, the eye tracker is operated to collect gaze data and, in some aspects, other relevant data, such as pupil size. One example of such a task is reading a paragraph. Another example of such a task is identifying a specific image among a set of images in response to an instruction or question. Tasks may include real life, everyday tasks for people to perform.

In aspects, the system 100 may include a second sensor 142 (FIG. 1) configured to measure a biomarker. In aspects, the processor 130 may measure, by the second sensor 142, the biomarker of the user while performing the task and prior to the treatment. For example, the biomarker may include a heart rate, a head movement, and/or fidgeting, among other bio markers.

Next, at step 208, the user is treated with a treatment. Treatments may include medication and/or non-medical treatment such as exercise.

Next, at step 210, after the treatment, the processor 130 captures a second data set indicating a performance of the an equivalent task by the user (e.g., a post-treatment test). The second data set may include eye-tracking data, such as the total amplitude of saccade and number of saccades during performance of the task, post-treatment. For example, the user may read the a passage of equivalent length (FIG. 4B), prior to treatment, and the processor 130 will measure the user's eye movements while reading the passage, using the eye-tracking device. The post-treatment test may be performed after a portion of treatment and/or after the course of treatment.

In aspects, the processor 130 may measure, by the second sensor 142 (FIG. 1), the biomarker of the user while performing the task after the treatment. For example, the heart rate of the user may be measured using the second sensor, (e.g., a heart rate monitor). The disclosed technology provides the benefit of combining eye-tracking with another biomarker to assist in the determination of efficacy of treatment. In aspects, additional biomarkers may be used as either features and/or cofactors in the prediction models. In aspects, additional non-biomarker inputs such as room light, and/or user behavior may be used as either features and/or cofactors in the prediction models.

Next, at step 212, the processor 130 performs an analysis of the baseline test and the post-treatment test. In aspects, the processor 130 may predict a measure of efficacy of the treatment using a trained machine learning model based on the first data set and the second data set. For example, the data set from the baseline test and the data set from post-treatment test may be input into a trained machine learning model (such as a neural network). The trained model may generate an output representing the measure of the improvement and/or deterioration to the user's ability to perform the task due to the treatment. Advantageously, the disclosed technology does not diagnose mental or neurological conditions, instead it determines a measure of efficacy of treatment for the mental or neurological conditions. The measure of an improvement and/or deterioration to an ability of the user to perform the task due to the treatment of the user is a value (e.g., a score) that may indicate a value of and/or a change in an ability of the user to perform a task based on the treatment. Such value and/or change may include an improvement, a deterioration, and/or no change in the ability of the user to perform one or more tasks due to the treatment of the user.

In aspects, additional data such as demographic data (e.g., age, and/or gender) may be used in the analysis of the differences between the baseline test data set and the post-treatment test data set. For example, a user may be a 13-year-old female. The processor 130 may utilize this data as additional inputs to the machine learning model.

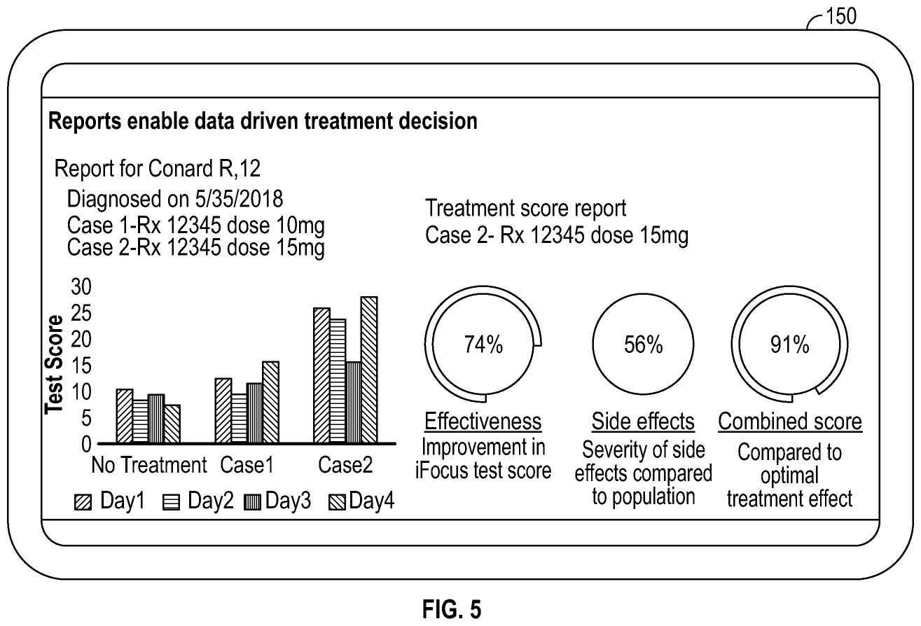

Next, the processor 130 outputs the measure of efficacy of treatment of the user to the clinician. In aspects, the processor 130 may display, on a display 150, a report (FIG. 5) configured to detail the results of the measure of efficacy of treatment of the user. For example, the report may show graphs comparing no treatment to different treatment options, such as a 10 mg dose of medication and/or exercise. The report may display the severity of side effects of the treatment.

For example, the processor 130 may display a recommended course of treatment based on the predicted measure of efficacy of treatment of the user. In aspects, the processor 130 may determine a recommended course of treatment based on a third dataset. In aspects, the third data set may be indicative of recommended courses of treatment. In aspects, various courses of treatment may be each assigned a value or a range of values. The predicted measure of efficacy of treatment of the user may be compared to the value or range of values. For example, exercise may be given a value range of 10-20, medication may be given a range of 0-10. If the predicted measure of the efficacy of treatment of the user is 13, the processor 130 may display a recommendation for exercise as an additional treatment. In aspects, the third dataset may be used to train a machine learning model to predict a recommended course of treatment.

In aspects, at step 214, the processor 130 may determine if additional treatment is required based on a period of time passing since last treatment, and or if there is a need to reassess treatment. A benefit is that the disclosed technology analyzes changes in performance.

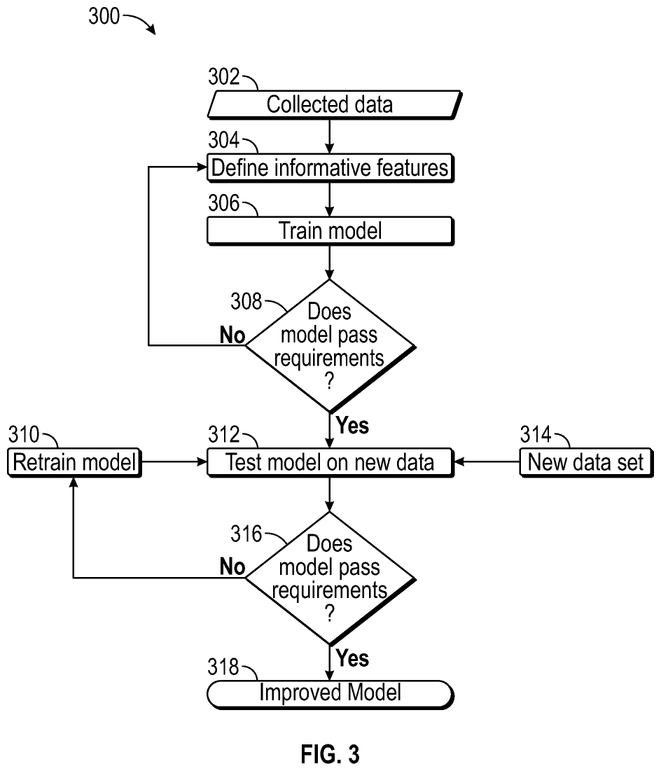

Referring to FIG. 3, the training process 300 of the machine learning model is shown. Initially, at step 302, the machine learning model is built by collecting data from multiple users, which were tested with and without treatment. The machine learning model is trained (306, 312) to differentiate between treated and untreated subjects using machine learning methods. In some aspects, the features which build the algorithm are from two sources. For example, one source may be features known from literature to be affected in, for example, ADD and/or ADHD users, such as saccade timing and amplitude and fixation location and timing. The second source are different features detected by the sensor (e.g., an eye tracker) and identified by data analysis tools to be significant in the model development, as illustrated in FIG. 3.

For example, the amount of eye movement or certain types of eye movement that a person engages in when reading a paragraph displayed on a screen may be determined prior to administration of a medicine and at a suitable time after administration (to provide sufficient time for the medicine to take effect). The two sets of data may be compared to generate a metric or other form of evaluating the impact of the medicine on the person's eye movements. Differences in the eye movement data may be attributed at least in part to the medicine, and in some cases, this may indicate an improved ability to perform the task (as indicated by a score or time to perform the task, etc.) or otherwise limit the impact of certain behaviors associated with, for example, ADHD.

Other tasks may include tasks requiring the user to keep their gaze at one location or follow a point or follow other instructions regarding gaze response to triggers on the screen. In these examples, the time direction and accuracy of response would be measured by looking at fixation and saccade data. Other tasks may include watching a video or playing a game on the screen, the pattern of eye movement, location of gaze, and/or other metrics may be measured.

In one aspect or implementation, the machine learning model may use one or more features extracted from the eye-tracking data; these may be determined based on statistical analysis showing a correlation of a change in the feature with a treatment, or by the impact of varying the features used in training a model on the accuracy of the output. As examples, the features may include one or more of saccade responses (timing, frequency, and amplitude, direction), fixation (duration and location), vergence (angle between the eyes), gaze location compared to specific areas of interest (AOI), frequency of blinks, smoothness of gaze movement. In the task example of reading a paragraph, features such as speed of reading, movement directions, total amplitude of saccade and number of saccades may be used.

Informative features 304 may be defined and used to train the machine learning model 306. The model may be tested to determine if it passes any testing requirements 308. In step 312, the trained machine learning model may be tested using new data 314 and retested in step 316 to see if the trained machine learning model passes the requirements. Examples of such requirements may include thresholds for one or more of: sensitivity, specificity, precision, recall, and/or area under an ROC curve (AUC).

Additional data 314 may enable adding more features and increasing the sensitivity and versatility of the algorithm/machine learning model. At some point, it is possible that the input to the algorithm or machine learning model may include all raw gaze data. The algorithm, machine learning model, or other form of data processing may be implemented in the form of a trained machine learning model in which a set of feature data representing characteristics of a subject person are classified with regards to whether they indicate a level of improvement at a task, efficacy of a treatment, or a lack of efficacy, for example.

In one aspect, the output of the algorithm or machine learning model may be provided to a clinician responsible for the user treatment and/or to the users and is expected to enable data-based treatment decisions. As an example, the output may be categorized as a positive, a neutral, and/or a negative treatment effect or may be treatment efficacy as reflected by a scale (such as 1-10).

In some aspects, the disclosed technology may be used to assess a user's sensitivity to change of testing or task performing conditions, such as time of day, ambient light, noise level, etc. In some aspects, the disclosed technology may be used to collect continuous eye-tracking data to provide real-time feedback to teachers/parents, and/or users. In some aspects, the disclosed technology can be used for differential diagnosis of ocular and vision problems or learning disability that might be perceived as ADHD, for example. In some aspects, accumulated data (and modeling, such as machine learning modeling) of the responses of different users to a range of treatments may enable the disclosed technology to provide not only feedback about treatment efficacy but also a recommendation regarding how to change a treatment to benefit a specific person or set of people. In some aspects, the disclosed technology may be used to assist in the evaluation or treatment of other neurological or mental conditions, such as cognitive impairment due to aging or injury, concussion, or substance abuse, etc. In some aspects, the disclosed technology may be used to assist in identifying the existence and predict a possible episode of depression or bipolar disease, as well as identify dementia, Multiple Sclerosis (MS), schizophrenia or other mental or neurological conditions. In aspects, the disclosed technology may be used to detect an onset of an episode of depression or bipolar disorder by monitoring different aspects of the user's interaction with the computer. The different aspects may include, for example, eye-tracking during different tasks, and/or typing patterns.

In aspects, the disclosed technology may measure a change in performance compared to a baseline. The outcome may include a measure of treatment efficacy if the measurement was performed relative to a specific treatment, or a measure of changes due to other factors—such as time of day, nutrition change, or hormonal change. For example, the processor 130 may identify a task, and prior to an event and while the user performs the task, measure the eye movements of the user with the sensor. After the event and while the user performs the task, the processor 130 may measure eye movements of the user with the sensor. The processor 130 may determine a difference between the eye movements of the user prior to and after the event based on a trained machine learning model and determine a measure of change in the condition based on the determined difference in the measured eye movements of the user. The processor 130 may then display a recommended a course of treatment for the user based on the determined measure of change. An event may include, for example, treatment, time of day, nutrition change, and/or hormonal change.

Nonlimiting examples of uses of the disclosed technology may include a web based a wellness application that uses eye movement to track concentration improvement compared to a baseline defined by the user; a product that consists of the platforms: a) Computer-based tool that tracks the users' eyes during tasks such as reading b) a companion application on a mobile device that collects input from the user regarding medication time and type, side effects and subjective performance c) An output tracking platform for the clinician; a system that tracks performance over time using eye-tracking and other biomarkers and inputs, that system measures change in performance and can inform the user regarding continuing or changing their current behavior; and a system that tracks changes of performance for people with additional mental issues such as depression and bio polar disorder. The system can identify the onset of an episode.

FIG. 4A illustrates an example of a displayed paragraph shown to a user/user in a proof-of-concept study or investigation for the disclosed technology. The circled area within the display represents a fixation point 402 of the user's focus within the displayed paragraph as a pretreatment task. The elongated line extending across words of the displayed paragraph represents a saccade 404 of the user, pretreatment.

In the investigation, the eye-tracking device (sensor 140) was mounted on a screen 150. In one test scenario, the users included children aged 12 and over and adults with an ADHD diagnosis, and who were regularly taking ADHD medication following a prescription from their clinician. Each user was tested once without medication and once after taking medication (the timing of the test was selected to be within the time interval in which the user thought the medication had the highest effectiveness, so this aspect was somewhat subjective). All users were regularly reading English at a middle school level or above. The tests consisted of a paragraph at a middle school reading level. There were two variations of the test such that a user would not see the same paragraph during the different tests.

FIG. 4B illustrates the same example displayed paragraph shown to the user, but showing the post-treatment saccades. The circled areas within the display represent the fixation point 402 of the user's focus within the displayed paragraph as a post-treatment task. The elongated line extending across words of the displayed paragraph represents a saccade 404 of the user, post-treatment.

In the investigation, saccades and fixations were identified, as well as the location of the person's gaze. These results were then analyzed statistically using the disclosed technology. In one example, the analysis consisted of statistical analysis of different features, such as total amplitude of saccades, number of saccades, length of fixation, as well as analysis of reading speed and efficiency (time spent re-reading).

The performance of each user with and without medication was then compared. Clear trends were observed in which the total amplitude of saccades and the number of saccades were both larger without medication compared to the same user with medication. The experiment also determined differences in reading speed and reading efficiency between pre- and post-treatment.

In aspects, the user may enter, on a user interface, the medication used for treatment, r side effects, and/or their personal assessment of efficacy.

The disclosed technology has one or more of the following differentiating advantages compared to conventional approaches: measurement process—conventional existing approaches or assessments rely on tasks in which people with ADHD, for instance, are expected to achieve a lower score than the general population. This approach has limitations: 1) Performance on specific tasks can improve if a test is repeated multiple times. People prefer to avoid tasks in which they expect they will perform poorly. A product that relies on users repeating a task they struggle with is likely to receive low adherence and may have lower reliability as a tool for evaluating treatments. 2) Comparing a user's performance to themselves instead of comparing to the general population, as done by the disclosed technology, is the most accurate way to measure treatment efficacy, as it is personalized, and experimental factors may be controlled or accounted for.

Other advantages or benefits of the approach described herein may include: measurement of biomarkers which the users can not consciously alter or control—this serves to overcome a possible suspicion of users intentionally underperforming; low-cost setup which enables at-home testing, so that no clinician is required during testing or for analysis; learning algorithm, the system can evolve to adapt to new data; and a short test which is designed to be repeated—this enables accurate progress measures as well as repeat testing to account for variability in a user's performance which might not be related to treatment.

Referring to FIG. 5, a diagram illustrating an exemplary report generated by the system of FIG. 1 is shown. The report may be displayed on display 150. The report may be configured to detail graphical and/or text representations of the results of the measure of the efficacy of treatment of the user. For example, the report may show bar and/or circle graphs comparing no treatment to different treatment options, such as a 20 mg dose of medication to a 10 mg dose, to exercise. The report may also display the displaying a recommended course of treatment for the user based on the determined measure of efficacy.

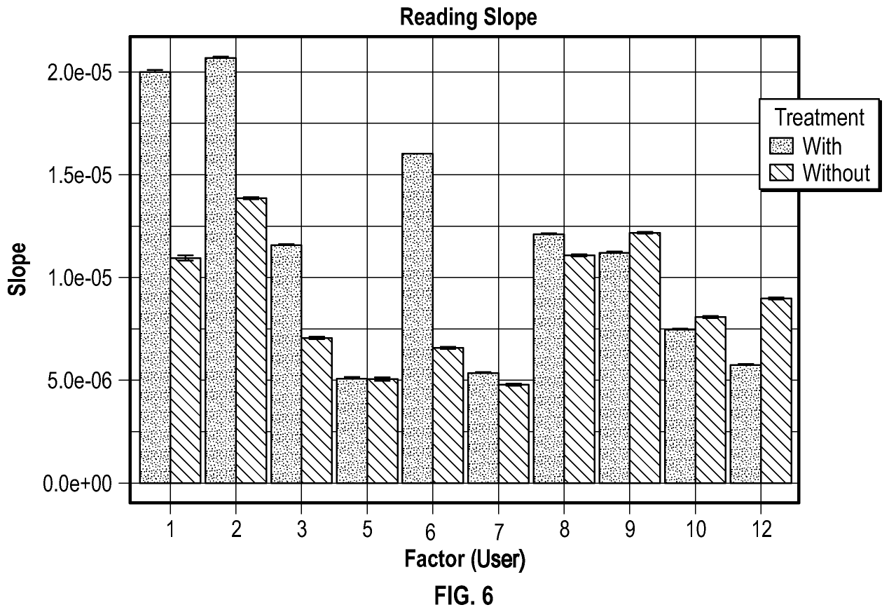

FIG. 6 shows a graphical representation of reading slope with and without user treatment. The disclosed analysis calculates the reading pace with and without treatment, by calculating the slope of the advancement along a y-axis of a page while reading several paragraphs. For each user, the first 100 time points were removed to remove noise related to the beginning and end of the reading session. A linear regression was calculated for the y axis points versus time. The slope was extracted for each user, for each test session—once with and once without treatment. The average slope for all users was found significantly higher with medication, indicating an overall faster reading pace.

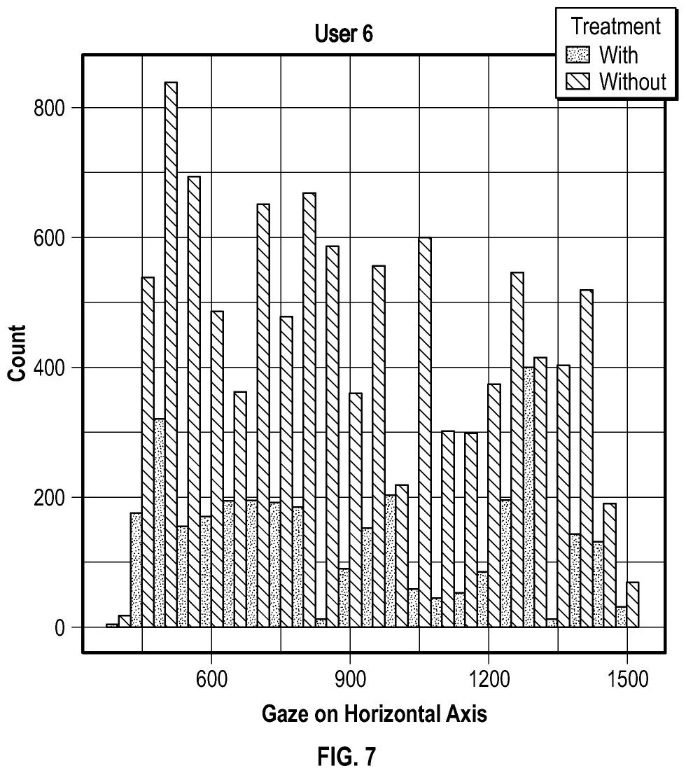

With reference to FIG. 7 a graphical representation of a number of times a page was re-read vs gaze with and without treatment is shown. A proposed reason for the lower reading ability and speed of users with ADHD is the need to re-read the same region in a given text. In theory, treatment (such as exercise and/or medication) can reduce this pattern. To test this hypothesis, the number of times on each page position on the X-axis was counted with and without treatment for each user, e.g., how many “lines” did the user truly read. Increased backtracking was detected in nearly all users in their untreated state, and treatment reduced the need to re-read text.

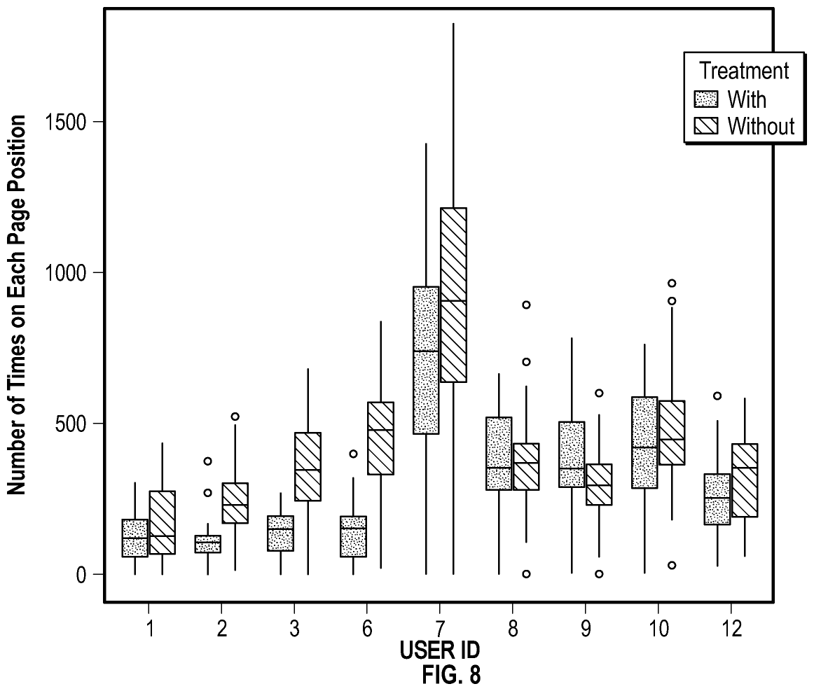

FIG. 8 shows a graphical representation of user gaze on each page position with and without user treatment. The distribution of the number of times each position in a page was visited for each user is shown as a box-plot in FIG. 8. Although there is a large difference between the re-read count of the different users, in many cases, a user re-reads regions more without treatment than with treatment.

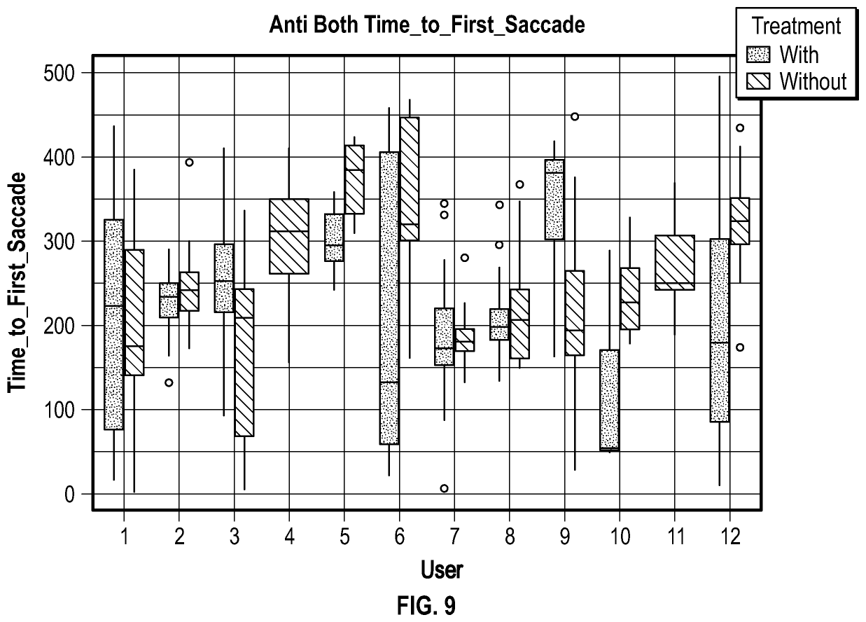

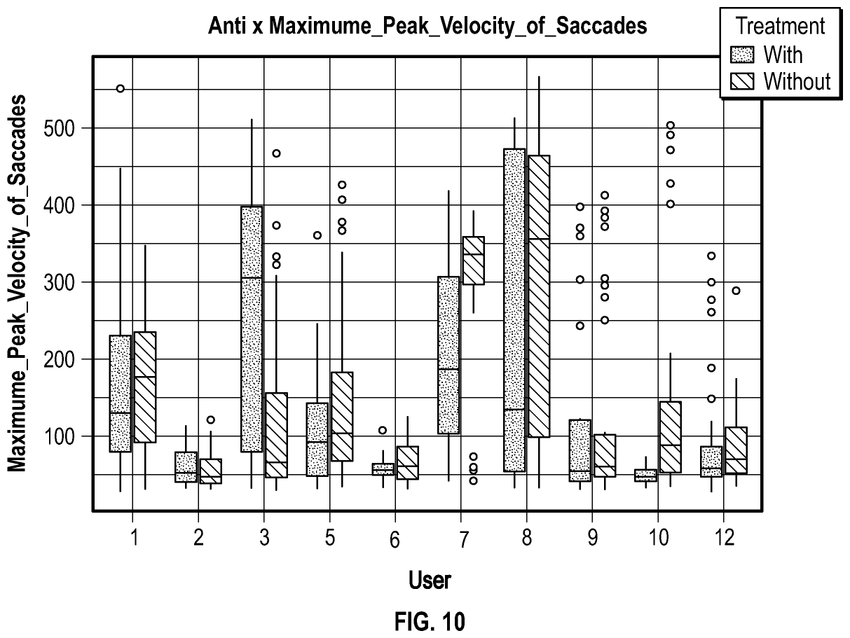

With reference to FIG. 9, a graphical representation of time to first saccade with and without user treatment (all users). Anti-saccades are an established marker for ADHD symptom severity. However, fewer of the ten users showed a difference between medicated and unmedicated states compared to the reading task. Four of the ten users showed an effect on time to first saccade compared to reading slope ( 6/10) and backtracking ( 5/10). The characteristics of the saccades were also investigated by measuring the amplitude and time of the saccade to calculate the max peak velocity reached during the saccade. Two of the four users that showed lower latencies for anti-saccades also showed reduced maximum peak velocities. The other two users that showed a reduced time of anti-saccades in response to medication showed no difference in saccade velocity, and one user exhibited reduced maximum peak velocity of anti-saccades that did not show a change in the anti-saccade response time. Overall, these results show differences in eye motion in ADHD users with and without medication but that the differences in eye motion vary from individual to individual, leading to an ensemble approach for algorithmic development that takes many features of eye motion into account. FIG. 10 shows a graphical representation of a maximum peak velocity of saccades with and without user treatment.

The disclosed structure may also utilize one or more controllers to receive various information and transform the received information to generate an output. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, Python, R, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

Persons skilled in the art will understand that the structures and methods specifically described herein and illustrated in the accompanying figures are non-limiting exemplary aspects, and that the description, disclosure, and figures should be construed merely as exemplary of particular aspects. It is to be understood, therefore, that this disclosure is not limited to the precise aspects described, and that various other changes and modifications may be effectuated by one skilled in the art without departing from the scope or spirit of the disclosure. Additionally, it is envisioned that the elements and features illustrated or described in connection with one exemplary aspect may be combined with the elements and features of another without departing from the scope of this disclosure, and that such modifications and variations are also intended to be included within the scope of this disclosure. Indeed, any combination of any of the disclosed elements and features is within the scope of this disclosure. Accordingly, the subject matter of this disclosure is not to be limited by what has been particularly shown and described.

What is claimed is:

1. A system for evaluating a treatment of a user for attention deficit hyperactivity disorder (ADHD), the system comprising:
a sensor configured to measure eye movements of the user while the user performs a task;
a processor; and
a memory including instructions, which, when executed by the processor, cause the system to:
identify a task;
prior to the treatment of the user and while the user performs the task, measure the eye movements of the user with the sensor;
while the user is under an effect of the treatment of the user and while the user performs the task, measure eye movements of the user with the sensor;
determine a difference between the eye movements of the user prior to and after the treatment of the user based on a trained machine learning model;
determine a measure of an efficacy of the treatment of the user based on the determined difference in the measured eye movements of the user; and
display a recommended a course of treatment for the user based on the determined measure of efficacy.

2. The system of claim 1, further comprising a second sensor configured to measure a biomarker, and
wherein the instructions, when executed by the processor, further cause the system to:
measure, by the second sensor, the biomarker of the user while the user performs the task and prior to the treatment of the user; and
measure, by the second sensor, the biomarker of the user while performing the task after the treatment.

3. The system of claim 2, wherein the instructions, when executed by the processor, further cause the system to determine a difference between the measured biomarker of the user prior to and after the treatment of the user.

4. The system of claim 3, wherein determining the measure of the efficacy of the treatment of the user is further based on the difference in the measured biomarker of the user.

5. The system of claim 3, wherein the biomarker includes at least one of a heart rate, a head movement, or fidgeting of the user.

6. The system of claim 1, wherein the task includes a section of text displayed on a screen for the user to read.

7. The system of claim 1, wherein the sensor includes an eye-tracking device.

8. The system of claim 1, wherein the instructions, when executed by the processor, further cause the system to:
determine the measure of the efficacy of the treatment of the user based on the determined difference in the measured eye movements of the user by inputting data relating to the eye movements of the user into the trained machine learning model, the trained machine learning model configured to generate an output representing a measure of change in an ability of the user to perform the task due to the treatment of the user, the change compared to the measured eye movements of the user prior to the treatment of the user.

9. The system of claim 8, wherein the data regarding the eye movements includes total amplitude of saccade and number of saccades during performance of the task.

10. A computer-implemented method for evaluating a treatment for a condition of a user, the computer-implemented method comprising:
accessing a first data set indicating a pretreatment condition;
accessing a second data set indicating a post-treatment condition;
predicting a measure of efficacy of the treatment of the user by a trained machine learning model based on the first data set and the second data set; and
displaying a recommended course of treatment for the user based on the predicted measure of efficacy.

11. The computer-implemented method of claim 10, wherein the first data set includes measured eye movements of the user while the user performs a task and prior to the treatment of the user, and wherein the second data set includes measured eye movements of the user while the user performs the task after the treatment of the user.

12. The computer-implemented method of claim 11, wherein the first data set further includes a measured biomarker of the user while the user performs the task and prior to the treatment, and wherein the second data set includes the measured biomarker of the user while the user performs the task after the treatment.

13. The computer-implemented method of claim 12, further comprising determining a difference between the measured biomarker of the user prior to and after the treatment of the user.

14. The computer-implemented method of claim 13, wherein predicting the measure of the efficacy of the treatment of the user is further based on the difference in the measured biomarker of the user.

15. The computer-implemented method of claim 12, wherein the biomarker includes at least one of a heart rate, a head movement, or fidgeting of the user.

16. The computer-implemented method of claim 11, wherein the task includes a section of text displayed on a screen for the user to read.

17. The computer-implemented method of claim 11, wherein the eye movements of the user are measured using an eye-tracking device.

18. The computer-implemented method of claim 11, further comprising:
predicting the measure of efficacy of the treatment of the user by the machine learning model based on the first data set and the second data set by inputting data relating to eye movements into the trained machine learning model, the trained model configured to generate an output representing a measure of change in an ability of the user to perform the task due to the treatment of the user, the change compared to the measured eye movements of the user prior to the treatment of the user.

19. The computer-implemented method of claim 10, further comprising:
comparing the prediction to a third data set indicative of recommended courses of treatment; and
determining the recommended course of treatment based on the comparison to the third data set.

20. A system for evaluating a change in a condition of a user for attention deficit hyperactivity disorder (ADHD), the system comprising:
a sensor configured to measure eye movements of the user while the user performs a task;
a processor; and
a memory including instructions, which, when executed by the processor, cause the system to:
identify a task;
prior to an event and while the user performs the task, measure the eye movements of the user with the sensor;
after the event and while the user performs the task, measure eye movements of the user with the sensor;
determine a difference between the eye movements of the user prior to and after the event based on a trained machine learning model; and
determine a measure of change in the condition based on the determined difference in the measured eye movements of the user.
